(19) United States
(12) Patent Application Publication
(10) Pub. No.: US 2021/0369091 A1
(43) Pub. Date: Dec. 2, 2021

(54) DISPOSABLE LIGHT HANDLE FOR ENDOSCOPY

(71) Applicant: 3M Endoscopy LLC, Lisle, IL (US)

(72) Inventors: Michael David Moen, Lisle, IL (US); Marcus Robert Moen, Lisle, IL (US); Matthew Ryan Moen, Lisle, IL (US)

(21) Appl. No.: 17/122,885

(22) Filed: Dec. 15, 2020

Related U.S. Application Data
(60) Provisional application No. 62/948,051, filed on Dec. 13, 2019.

(51) Int. Cl. A61B 1/00; F21V 23/04; F21S 9/02; F21V 29/76; F21V 29/89; A61B 1/06; A61B 1/12

(52) U.S. Cl. CPC A61B 1/00103; F21V 23/04; F21S 9/02; F21V 29/76; F21Y 2115/10; A61B 1/0684; A61B 1/00126; A61B 1/128; A61B 1/00105; F21V 29/89

(57) ABSTRACT

A single-use sterile light source apparatus for endoscopy is provided. One example apparatus includes a main body acting as casing, a connection port configured to attach to an endoscope, a LED light emitter capable of producing light with different intensities, at least one circuit board coupled to the LED light emitter, a non-rechargeable battery unit, and a power switch for controlling the intensity of the LED light emitter.

### CROSS REFERENCE TO RELATED APPLICATIONS

This application claims priority to and the benefit of U.S. Provisional Application No. 62/948,051 filed on Dec. 13, 2019, the entire content of which are incorporated by reference herein for all purposes.

### TECHNICAL FIELD

This specification describes examples of a disposable, single use, cordless light source for endoscopy procedures.

### BACKGROUND

Endoscopy in the medical field allows internal features of the body of a patient to be viewed without the use of traditionally, fully-invasive surgical procedures. Endoscopes, used in endoscopy, can be rigid or flexible with the distal portion designed to be inserted into the patient and the proximal end containing an eyepiece to allow the user to visualize the operative field. Many endoscopy systems utilize a camera which can be placed over the eyepiece and is connected to a video display monitor to allow visualization of the operative field by multiple individuals in the operating/procedure room. Endoscopes can be inserted through small incisions or through a natural body orifice and have different names depending on where the endoscope is used, for example, arthroscope (joint) cystoscope (bladder), laparoscope (abdomen), and colonoscope (colon).

In order to illuminate the body cavity in which the endoscope is inserted, a light delivery system is required. In general, the light source is connected to a light port of the endoscope which is located near the eyepiece at the proximal end of the endoscope. The light source used by most endoscopy systems include high power white light sources such as xenon lamps.

A light cord with fiber-optic cables is often used to connect a generator box of the light source to the endoscope. The light generator box is usually not sterile and is located at a certain distance from the patient and the endoscope. To allow for placement in the sterile environment in which endoscopy procedures often take place in, the light cord must be sterilized. However, the end of the light cord that connects to the generator box is not sterile. Therefore, the use of light cords introduces the risk of contamination of the sterile field and subsequent infections for patients undergoing endoscopy procedures. Many light cords require various adapters to allow direct attachment of the light cord to the endoscope. For use in sterile environments, these adapters must be sterilized before being attached to the endoscope in order to maintain sterility. If the small adapter is lost, the light cord cannot be attached to the endoscope.

Additionally, one of the most important safety concerns related to the use of high power light sources is related to the heat generated by the light source. Generator boxes of high power light sources generate a significant amount of heat which is transferred to the end of the fiber optic light cord resulting in the potential for fires and burns for patient due to the high energy output at the end of the cord.

Furthermore, another significant drawback regarding the use of light cords for endoscopy procedures that require a sterile environment is that the light cords must be sterilized before each use. This limits the number of procedures that can be performed in any given amount of time due to the necessity of sterilizing the cords. Additionally, the process of sterilization is also expensive and repeated sterilization of the light cords result in degradation of the fiber-optic cables over time, resulting in inadequate light transmission requiring a replacement of the light cords.

Finally, the use of light cords during surgery is cumbersome due to the fact that one end of the cord is on the sterile field and the other end of the cord is attached to the non-sterile light source, which is typically mounted on a tower with other equipment used for endoscopy procedures such as a camera housing, monitor, insufflation equipment and a printer.

To address the above stated challenges, a cordless light source than can be attached to the endoscope is highly preferred. At present, cordless light sources, which are electrically charged or powered by batteries, are available. However, these cordless light sources are designed for repetitive use during non-sterile procedures in the medical office setting and are not suitable for single use during sterile procedures. Additionally, currently available cordless light sources typically usually only provide a single intensity of emitted light, limiting their use to specific procedures where the light intensity of the light source is sufficient to illuminate the space.

There exists a need for a single use, sterile cordless light source that can be used with an endoscope in a sterile field that is efficient and is safe for exposure to the patient. Additionally, there is also a need of the cordless light source to be able to provide different intensities of emitted lighted to broaden the use of light source for various types of endoscopic procedures.

### SUMMARY

In one embodiment, a single-use sterile light source apparatus for endoscopy is provided. The apparatus includes a main body acting as casing, a connection port configured to attach to an endoscope, a LED light emitter capable of producing light with different intensities, at least one circuit board coupled to the LED light emitter, a non-rechargeable battery unit, and a power switch for controlling the intensity of the LED light emitter.

In another embodiment, another single-use sterile light source apparatus for endoscopy is provided. The apparatus includes a main body acting as casing, a connection port configured to attach to an endoscope, a LED light emitter capable of producing light with different intensities, at least one circuit board coupled to the LED light emitter, a non-rechargeable battery unit, a heat sink surrounding the main body for heat dissipation, and a power switch for controlling the intensity of the LED light emitter.

### DESCRIPTION OF THE DRAWINGS

The accompanying drawings, which are incorporated in and constitute a part of the specification, illustrate various example methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. Furthermore, elements may not be drawn to scale.

FIG. 1A illustrates an exemplary schematic of the example endoscope system with an exemplary light source.

FIG. 1B illustrates an exemplary endoscope system set-up with the exemplary light handle as the light source in accordance with FIG. 1A.

FIG. 2A presents a perspective view of the exemplary light handle as the light source in accordance with FIG. 1A.

FIG. 2B presents a cross-sectional view of the exemplary light handle as the light source in accordance with FIG. 2A.

FIG. 3A presents a perspective view of another embodiment of the exemplary light handle as the light source in accordance with FIG. 1A.

FIG. 3B presents a cross-sectional view of the another embodiment of the exemplary light handle as the light source in accordance with FIG. 3A.

FIG. 4A presents a perspective view of a further embodiment of exemplary light handle as the light source in accordance with FIG. 1A.

FIG. 4B presents a cross-sectional view of the further embodiment of the exemplary light handle as the light source in accordance with FIG. 4A.

FIG. 4C presents a detailed perspective view of the further embodiment of the exemplary light handle as the light source in accordance with FIG. 4A.

### DETAILED DESCRIPTION

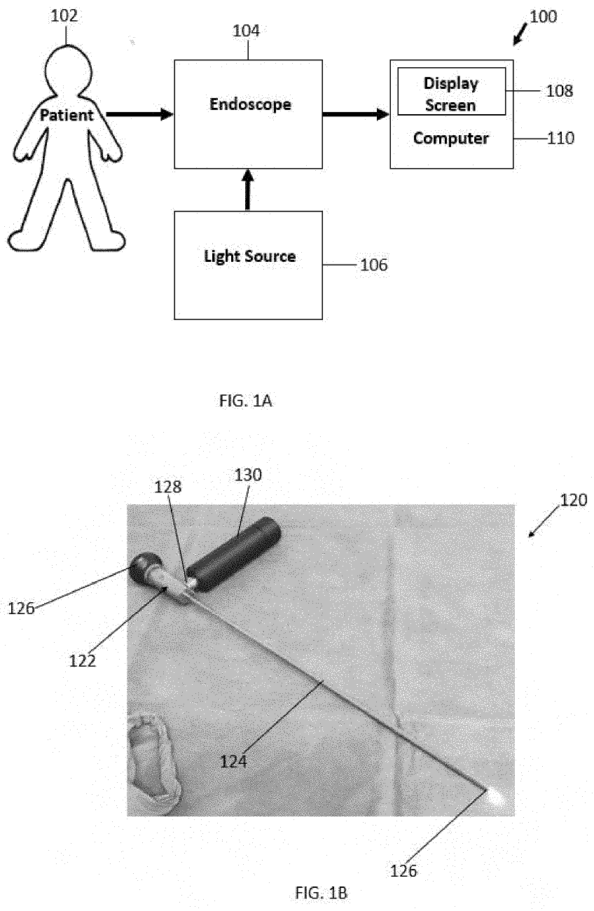

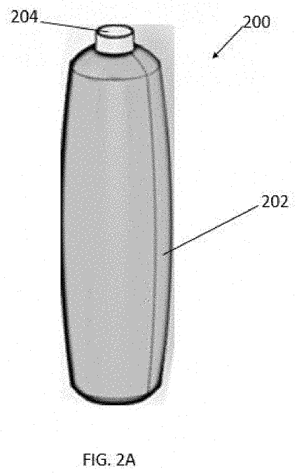

FIGS. 1A and 1B illustrate an exemplary schematic 100 and endoscopy system set-up 120 with an exemplary light handle as the light source in accordance with one illustrative embodiment. The exemplary schematic 100 depicted in FIG. 1A presents an endoscopy system set-up where an endoscope 104 is usually positioned in a targeted region on a patient 102. The positioning of the endoscope 104 is usually invasive and the endoscope 104 is generally inserted into a body opening (i.e., mouth and anus) or an in an incision made on a targeted region of the body of the patient 102. The endoscope 104 is connected to a light source 106 which supplies light of illumination for targeted region. The endoscope 104 is usually connected to a computer 110 in which the targeted region being viewed can be displayed as images on a display screen 108. The clinician performing the endoscopy procedure can further process the visualized images on the computer 110 and make digital annotations on display screen 108 to identify regions of interest.

The endoscope 104 depicted in FIG. 1A is configurable to be changeable between a normal imaging mode for imaging an object of interest, and a vessel enhancement imaging mode for enhancing and imaging blood vessels present in mucosa as objects of interest. When the endoscope is configured to be in the normal imaging mode, a normal multi-color image is produced by the computer 110 and displayed by the display screen 108. The normal multi-color image is generally suitable for general diagnosis of the object of interest. Additionally, in the vessel enhancement imaging mode, a vessel enhancement image is produced by the computer 110 and displayed by the display screen 108. The vessel enhancement image is suitable for diagnosing the pattern of blood vessels.

FIG. 1B illustrates an exemplary endoscope system set-up 120 with the exemplary light handle as the light source in accordance with one illustrative embodiment. In one embodiment, the endoscope system set-up comprises an endoscope 122. The endoscope 122 includes an elongated tube 124 with a distal end 126. The distal end 126 of the elongated tube 124 of the endoscope is entered into the in a body cavity of a patient 102. In some embodiments, the elongated tube 124 is flexible and can be controlled for movement electronically or mechanically. The endoscope 122 also comprises an image outlet port 126 which connects to the computer 110 to process and visualize the images captured through the elongated tube 126. In some embodiments, the image outlet port 126 is connected to the computer 110 via a universal cable (not shown in the figure). The endoscope 122 further includes a light port 128 configured to connect to a light source for illuminating the target region of interest through the elongated tube 124. In one embodiment described by the present disclosure, an exemplary light handle 130 is connected to the light port 128 acting as the light source for the endoscope 122.

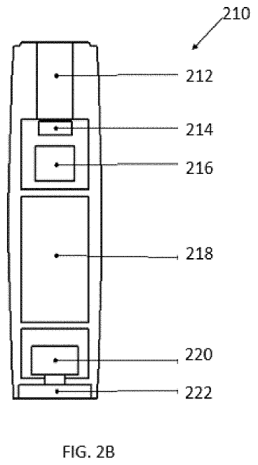

FIGS. 2A and 2B present a perspective view 200 and a cross sectional view 210 of the exemplary light handle 130 as the light source in accordance with one illustrative embodiment. In FIG. 2A, the perspective view 200 of one illustrative embodiment of the light handle 130 is depicted. In this embodiment, the light handle 130 has been made to be a stand-alone component that can be packaged in a sterile packaging and used a single-use attachment. The light handle 130 has a main body 202 for handholding and the main body 202 also acts as the casing to enclose all the components of the light handle 130. In some embodiments, the main body 202 is produced from a thermally conductive plastic for heat resistance as well as heat dissipation and comprises surface texturing for improved grip. In other embodiments of the light handle 130, the main body 202 is produced from an aluminum alloy. Additionally, the light handle 130 also has a connection port 204 to connect to the light port 128 of the light handle 130.

In reference to FIG. 2B, a cross sectional view 210 of the exemplary light handle 130 is presented in accordance with one illustrative embodiment. The connection port 204 of the light handle 130 includes a scope mounting adapter 212 to be able to secure the connection port 204 to the light port 128 of the light handle 130. In some embodiments of the present disclosure, the scope mounting adapter 212 is threaded to be able to be secured to the light port 128 of the light handle 130. The light handle also comprises a light-emitting diode (LED) light emitter 214 controlled by a circuit board 216. The LED light emitter 214 is aligned in the direction such that light passes through the scope mounting adapter 212 and into the light port 128 of the light handle 130. The LED light emitter 214 and the respective circuit board 216 is powered by a non-rechargeable battery unit 218 encompassed in the light handle 130. To control the intensity of the light produced by the LED light emitter 214, the light handle 130 further includes a power switch 222 connected to another circuit board 220 for communicating with the circuit board 216 of the LED light emitter 214. In the above stated embodiment of the light handle 130, the power switch 222 is designed to be turned in a circumferential fashion with one hand of the clinician to increase the intensity of the LED light emitter 214 while the light handle 130 is held by the other hand of the clinician.

The LED light emitter 214 comprises several LEDs and each of the LEDs has a p-type semiconductor and an n-type semiconductor attached together in a fashion that is well-known in the art. Upon application of voltage from the battery unit 218, recombination of a positive hole and an electron occurs across the band gap at the p-n junction, for a current to flow. Light is emitted by generation of energy according to the band gap. The light amount emitted by the LEDs of the LED light emitter 214 is increased by an increase in supplied power by the battery unit 218, which in turn is controlled by the power switch 222 connected to the circuit board 220 for communicating with the circuit board 216 of the LED light emitter 214.

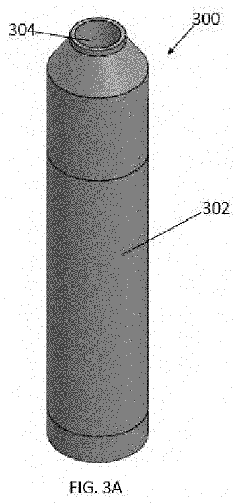

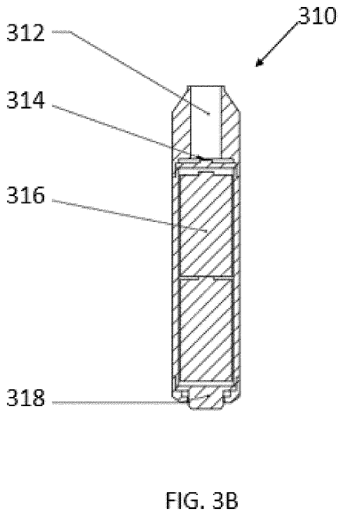

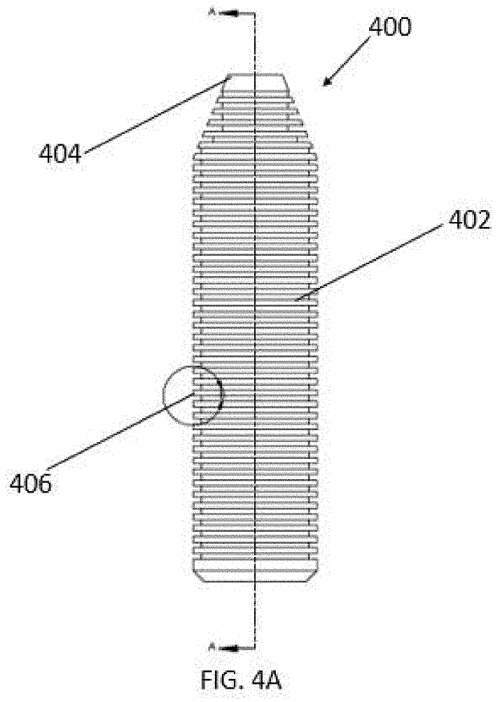

FIGS. 3A and 3B present a perspective view 300 and a cross sectional view 310 of the exemplary light handle 130 as the light source in accordance with another illustrative embodiment. In FIG. 3A, the perspective view 300 of another illustrative embodiment of the light handle 130 is depicted. In this embodiment, the light handle 130 has also been made to be a stand-alone component that can be packaged in a sterile packaging and used a single-use attachment. The light handle 130 has a main body 302 for handholding and the main body 302 also acts as the casing to enclose all the components of the light handle 130. In some embodiments, the main body 302 is produced from a thermal conductive metal such as an aluminum alloy for heat resistance as well as heat dissipation where the external temperature of the main body 302 is restricted to go beyond a temperature of 92° F. Additionally, in this embodiment of the light handle 130, the light handle 130 also has a connection port 304 to connect to the light port 128 of the light handle 130.

In reference to FIG. 3B, a cross sectional view 310 of the exemplary light handle 130 is presented in accordance with another illustrative embodiment. The connection port 304 of the light handle 130 includes a scope mounting adapter 312 to be able to secure the connection port 304 to the light port 128 of the light handle 130. In some embodiments of the present disclosure, the scope mounting adapter 312 is threaded to be able to be secured to the light port 128 of the light handle 130. In some embodiments of the present disclosure, the scope mounting adapter 312 is designed with a press-fit design to be able to be secured to the light port 128 of the light handle 130. The light handle also comprises a light-emitting diode (LED) light emitter circuit board combination 314. The LED light emitter circuit board combination 314 is aligned in the direction such that light passes through the scope mounting adapter 312 and into the light port 128 of the light handle 130. The LED light emitter circuit board combination 314 is powered by a non-rechargeable battery unit 316 encompassed in the light handle 130. To control the intensity of the light produced by the LED light emitter circuit board combination 314, the light handle 130 further includes a power switch 318 for communicating with the LED light emitter circuit board combination 314. In this described embodiment of the light handle 130, the power switch 318 is designed to be turned in a push-button fashion, in which the power switch 318 is pressed to a depressed configuration, the intensity of the LED light emitter circuit board combination 314 is changed. Repeatedly use of the push-button fashion of the power switch 318 cycles through various levels of intensity of the LED light emitter circuit board combination 314 with the use of only one hand of the clinician.

The LED light emitter circuit board combination 314 comprises several LEDs and each of the LEDs has a p-type semiconductor and an n-type semiconductor attached together in a fashion that is well-known in the art. Upon application of voltage from the battery unit 316, recombination of a positive hole and an electron occurs across the band gap at the p-n junction, for a current to flow. Light is emitted by generation of energy according to the band gap. The light amount emitted by the LEDs of the LED light emitter circuit board combination 314 is increased by an increase in supplied power by the battery unit 316, which in turn is controlled by the power switch 318 by communicating with the LED light emitter circuit board combination 314.

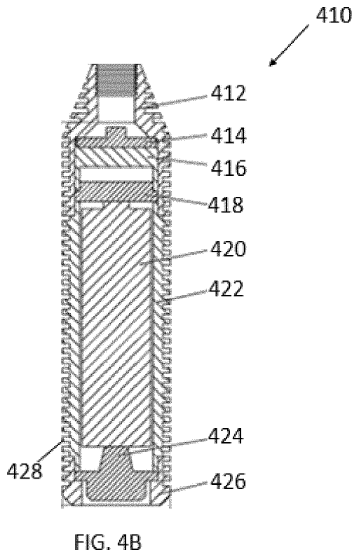

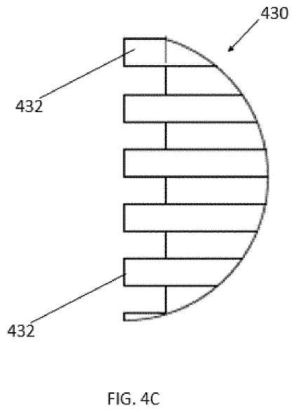

FIGS. 4A-4C present a perspective view 400, a cross sectional view 410, and a presents a detailed perspective view 430 of the further embodiment of the exemplary light handle 130 as the light source in accordance with further another illustrative embodiment. In FIG. 4A, the perspective view 400 of another further illustrative embodiment of the light handle 130 is depicted. In this described embodiment, the light handle 130 has also been made to be a stand-alone component that can be packaged in a sterile packaging and used a single-use attachment. The light handle 130 has a main body 402 for handholding and the main body 402 also acts as the casing to enclose all the components of the light handle 130. In some embodiments, the main body 402 is produced from a thermal conductive metal such as an aluminum alloy for heat resistance as well as heat dissipation where the external temperature of the main body 402 is restricted to go beyond a temperature of 92° F. The main body 402 is covered by a heatsink 406 throughout the entire surface of the main body 402. Additionally, in this embodiment of the light handle 130, the light handle 130 also has a connection port 404 to connect to the light port 128 of the light handle 130.

In reference to FIG. 4B, a cross sectional view 410 of the exemplary light handle 130 is presented in accordance with another illustrative embodiment. The connection port 404 of the light handle 130 includes a scope mounting adapter 412 to be able to secure the connection port 404 to the light port 128 of the light handle 130. In some embodiments of the present disclosure, the scope mounting adapter 412 is threaded to be able to be secured to the light port 128 of the light handle 130. In some embodiments of the present disclosure, the scope mounting adapter 412 is designed with a press-fit design to be able to be secured to the light port 128 of the light handle 130. The light handle also comprises a light-emitting diode (LED) light emitter combination 414. The LED light emitter 414 is aligned in the direction such that light passes through the scope mounting adapter 412 and into the light port 128 of the light handle 130. To prevent any movement of the LED light emitter 414 within the light handle 130, the LED light emitter 414 is held securely in place by a retainer 416. The LED light emitter 414 is controlled by a LED driver 418 and the LED light emitter 414 is powered by a non-rechargeable battery unit 420 securely encompassed in the body compartment 422 of light handle 130. To control the intensity of the light produced by the LED light emitter 414, the light handle 130 further includes a power switch 424 for communicating with the LED driver 418 and LED light emitter 414. In this described embodiment of the light handle 130, the power switch 424 is designed to be turned in a push-button fashion and housed in a light button compartment 426 to prevent the accidental pressing of the power switch 424 in midst of a sterile procedure. When the power switch 424 is pressed to a depressed configuration, the intensity of the LED light emitter 414 is changed. Repeatedly use of the push-button fashion of the power switch 424 cycles through various levels of intensity of the LED light emitter 414 with the use of only one hand of the clinician.

The LED light emitter 414 also comprises several LEDs and each of the LEDs has a p-type semiconductor and an n-type semiconductor attached together in a fashion that is well-known in the art. Upon application of voltage from the battery unit 420, recombination of a positive hole and an electron occurs across the band gap at the p-n junction, for a current to flow. Light is emitted by generation of energy according to the band gap. The light amount emitted by the LEDs of the LED light emitter 414 is increased by an increase in supplied power by the battery unit 316, which in turn is controlled by the power switch 424 by communicating with the LED light emitter 414.

FIG. 4C presents a detailed perspective view 430 of the further embodiment of the exemplary light handle as the light source in accordance with another illustrative embodiment. To increase dissipation of heat from the main body 402 of the light handle 130, the entire surface of the main body 402 is surrounded by a heat sink 406. In the detailed perspective view 430, the heat sink 406 comprises a plurality of radial cooling fins 432 which protrude away from the surface of the main body 402 radially, increasing the surface area for heat dissipation and ensuring thermal control of the LED light emitter 414. In one embodiment of the present disclosure, the radial cooling fins 432 are each 1.5 millimeters (mm) tall, 1 mm wide and have a spaced 1 mm apart. In another embodiment of the present disclosure, the radial cooling fins 432 are produced from thermally conductive materials such as aluminum alloys and are produced by processes such as extrusion, casting, skiving, or miling.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 724 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A single-use sterile light source apparatus for endoscopy, the apparatus comprising:
a main body acting as casing;
a connection port configured to attach to an endoscope;
a LED light emitter capable of producing light with different intensities;
at least one circuit board coupled to the LED light emitter;
a non-rechargeable battery unit; and
a power switch for controlling the intensity of the LED light emitter.

2. The apparatus according to claim 1, wherein the main body is produced from a thermal conductive plastic.

3. The apparatus according to claim 1, wherein the main body is produced from an aluminum alloy.

4. The apparatus according to claim 1, wherein the connection port further comprises a scope mounting adapter to attach to the endoscope.

5. The apparatus according to claim 4, wherein the scope mounting adapter comprises a press-fit design.

6. The apparatus according to claim 1, wherein the power switch is designed to rotate in a circumferential pattern.

7. The apparatus according to claim 1, wherein the power switch is designed to be a push-button.

8. The apparatus according to claim 7, wherein the push-button design of power switch is configured to change intensities of the LED light emitter.

9. A single-use sterile light source apparatus for endoscopy, the apparatus comprising:
a main body acting as casing;
a connection port configured to attach to an endoscope;
a LED light emitter capable of producing light with different intensities;
at least one circuit board coupled to the LED light emitter;
a non-rechargeable battery unit;
a heat sink surrounding the main body for heat dissipation; and
a power switch for controlling the intensity of the LED light emitter.

10. The apparatus according to claim 1, wherein the heat sink comprises of a plurality of cooling fins.

11. The apparatus according to claim 10, wherein the plurality of the cooling fins are produced from an aluminum alloy.

12. The apparatus according to claim 12, wherein each of the plurality of the cooling fins has a height of 1.5 mm, width of 1 mm, wherein the plurality of cooling fins are spaced 1 mm apart.
